(19) United States
(12) Patent Application Publication
(10) Pub. No.: US 2009/0258888 A1
(43) Pub. Date: Oct. 15, 2009

(54) Pyrimidine Derivatives

(75) Inventors: Dhanapalan Nagarathnam, Bethany, CT (US); Yuanwei Chen, North Haven, CT (US); Wenlang Fu, Milford, CT (US); Donald Bierer, Bethany, CT (US); Ming Wang, Belle Mead, NJ (US); Michael Brands, Wuppertal (DE); Yamin Wang, Sandy Hook, CT (US); Brian R. Bear, Oceanside, CA (US); Aaron Schmitt, Hamden, CT (US); Eric Mull, Guilford, CT (US); David Miller, Waltham, MA (US); Jin Zhao, Natick, MA (US)

Correspondence Address: EDWARDS ANGELL PALMER & DODGE LLP, P.O. BOX 55874, BOSTON, MA, 02205, US

(73) Assignee: Bayer Pharmaceuticals Corporation, West Haven, CT (US)

(21) Appl. No.: 11/918,016

(22) PCT Filed: Apr. 7, 2006
(86) PCT No.: PCT/US06/13505

§ 371 Date: Jun. 16, 2009

Related U.S. Application Data
(60) Provisional application No. 60/669,462, filed on Apr. 8, 2005.

(51) Int. Cl. A61K 31/506; C07D 239/46; A61P 35/00

(52) U.S. Cl. 514/275; 544/297

(57) ABSTRACT

This invention relates to novel compounds and processes for their preparation, methods of treating diseases, particularly cancer, comprising administering said compounds, and methods of making pharmaceutical compositions for the treatment or prevention of disorders, particularly cancer.

This invention relates to novel compounds and processes for their preparation, methods of treating diseases, particularly cancer, comprising administering said compounds, and methods of making pharmaceutical compositions for the treatment or prevention of disorders, particularly cancer.

Nitrogen-containing heterocycles such as pyrimidine derivatives have been disclosed in patent and non-patent publications as having a variety of pharmaceutical properties and utilities. Several such publications are listed below.

WO 03/062225 (Bayer) relates to pyrimidine derivatives as rho-kinase inhibitors, and their use in treatment of rho-kinase mediated conditions including cancer.

WO 2001/87845 (Fujisawa) relates to N-containing heterocyclic compounds having 5-HT antagonistic activity. These compounds are stated as being useful for treating or preventing central nervous system disorders.

WO 95/10506 (Du Pont Merck) relates to 1N-alkyl-N-arylpyrimidinamines and derivatives thereof, which are stated to inhibit the corticopropin releasing factor (CRF) peptide and to be useful for treatment of psychiatric disorders and neurological diseases.

WO 2004/048365 (Chiron) relates to 2,4,6-trisubstituted pyrimidines as phosphotidylinositol (PI) 3-kinase inhibitors and their use in treatment of cancer. WO 2004/000820 (Cellular Genomics) relates to N-containing heterocycles and other compounds as kinase modulators, and their use in treatment of numerous kinase-associated disorders including cancer.

WO 01/62233 (Hoffmann La Roche) relates to nitrogen-containing heterocycles and their use in treatment of diseases modulated by the adenosine receptor.

US 2004/0097504 (Vertex) relates to nitrogen-containing heterocycles useful in treatment of various protein kinase-mediated disorders.

The pharmaceutical field is always interested in identifying new pharmaceutically active compounds. Such materials are the subject of the present application.

In one embodiment, the present invention provides a compound of formula (I)

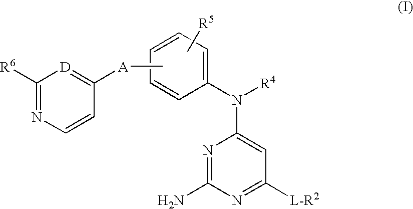

wherein
A represents an oxygen atom or a group —NR^{A}—, in which R^{A} represents hydrogen or alkyl;
D represents a group CH— or a nitrogen atom;
L is a 2 carbon atom linker selected from the group consisting of ethandiyl, ethendiyl and ethyndiyl, which in case of ethandiyl can optionally be substituted by 0, 1 or 2 alkyl, hydroxy or alkoxy, in case of ethendiyl can optionally be substituted by 0, 1 or 2 alkyl or alkoxy;
R² represents alkyl, wherein alkyl can be substituted with 0 to 3 substituents selected from the group consisting of halo, hydroxy, alkoxy, amino, alkylamino, and alkylsulfonylamino; or
R² represents phenyl or heteroaryl, wherein phenyl or heteroaryl can optionally be substituted by 0, 1 or 2 substituents selected from the group consisting of halo, trifluoromethyl, alkyl, hydroxy, alkoxy, amino, alkylcarbonylamino, alkylamino, aminocarbonyl, alkylaminocarbonyl, aminosulfonyl, alkylaminosulfonyl, and,
wherein said alkylamino, alkylaminocarbonyl, and alkylaminosulfonyl are optionally substituted by 0, 1 or 2 substituents selected from the group consisting of hydroxy, halogen and alkoxy;
   R⁴ is hydrogen or alkyl;
   R⁵ is hydrogen or halo; and

R⁶ represents alkyl, cyano, aminocarbonyl, alkylaminocarbonyl, trifluoromethyl, amino, alkylcarbonylamino, alkylcarbonyl, alkenyl, alkynyl or chloro;

or a pharmaceutically acceptable salt thereof.

In another embodiment, the present invention provides a compound of formula (I), wherein

A represents an oxygen atom;
D represents a group —CH—;
L is a 2 carbon atom linker selected from the group consisting of ethandiyl, ethendiyl and ethyndyl;
R² represents alkyl, wherein alkyl can be substituted with 0 to 2 substituents selected from the group consisting of halo, hydroxy, alkoxy, amino, alkylamino, and alkylsulfonylamino; or
R² represents phenyl or pyridyl, wherein phenyl or pyridyl can optionally be substituted by 0, 1 or 2 substituents selected from the group consisting of halo, alkyl, hydroxy, and alkoxy;
R⁴ is hydrogen;
R⁵ is hydrogen; and
R⁶ represents alkyl, cyano, aminocarbonyl, chloro or trifluoromethyl;
or a pharmaceutically acceptable salt thereof.

In another embodiment, the present invention provides a compound of formula (Ia),

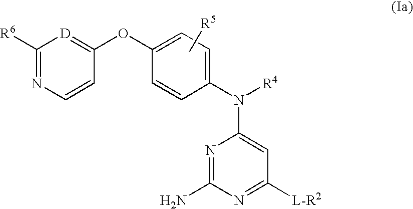

wherein
D represents a group —CH—;
L is a 2 carbon atom linker selected from the group consisting of ethandiyl, ethendiyl and ethyndyl;
R² represents phenyl or pyridyl, wherein phenyl or pyridyl can optionally be substituted by 0, 1 or 2 substituents selected from the group consisting of halo, alkyl, hydroxy, and alkoxy;
R⁴ is hydrogen;
R⁵ is hydrogen; and
R⁶ represents alkyl, cyano, aminocarbonyl, chloro or trifluoromethyl;
or a pharmaceutically acceptable salt thereof.

Depending on their structure, the compounds according to the invention can exist in stereoisomeric forms (enantiomers or diastereomers). The invention therefore relates to the enantiomers or diastereomers and to their respective mixtures. Such mixtures of enantiomers or diastereomers can be separated into stereoisomerically unitary constituents in a known manner.

Unless otherwise stated, the following definitions apply for the technical expressions used throughout this specification and claims:

Salts for the purposes of the invention are preferably pharmacologically acceptable salts of the compounds according to the invention.

Pharmaceutically acceptable salts of the compounds (I) include acid addition salts of mineral acids, carboxylic acids and sulfonic acids, for example salts of hydrochloric acid, hydrobromic acid, sulfuric acid, phosphoric acid, methanesulfonic acid, ethanesulfonic acid, toluenesulfonic acid, benzenesulfonic acid, naphthalenedisulfonic acid, acetic acid, propionic acid, lactic acid, tartaric acid, malic acid, citric acid, fumaric acid, maleic acid and benzoic acid.

Pharmaceutically acceptable salts of the compounds (I) also include salts of customary bases, such as for example and preferably alkali metal salts (for example sodium and potassium salts, alkaline earth metal salts (for example calcium and magnesium salts) and ammonium salts derived from ammonia or organic amines having 1 to 16 carbon atoms, such as illustratively and preferably ethylamine, diethylamine, triethylamine, ethyldiiso-propylamine, monoethanolamine, diethanolamine, triethanolamine, dicyclohexylamine, dimethylaminoethanol, procaine, dibenzylamine, N-methylmorpholine, dihydroabietylamine, argmine, lysine, ethylenediamine and methylpiperidine.

Alkyl represents a linear or branched alkyl radical having generally 1 to 6, 1 to 4 or 1 to 3 carbon atoms, illustratively representing methyl, ethyl, n-propyl, isopropyl, tert-butyl, n-pentyl and n-hexyl.

Alkenyl represents a linear or branched alkyl radical having one or more double bonds and 2 to 6, 2 to 4 or 2 to 3 carbon atoms, illustratively representing ethylene or allyl.

Alkynyl represents a linear or branched alkyl radical having one or more triple bonds and generally 2 to 6, 2 to 4 or 2 to 3 carbon atoms, illustratively representing propargyl.

Alkoxy represents a straight-chain or branched hydrocarbon radical having 1 to 6, 1 to 4 or 1 to 3 carbon atoms and bound via an oxygen atom, illustratively representing methoxy, ethoxy, propoxy, isopropoxy, butoxy, isobutoxy, pentoxy, isopentoxy, hexoxy, isohexoxy. The terms “alkoxy” and “alkyloxy” are often used synonymously.

Alkylamino represents an alkylamino radical having one or two (independently selected) alkyl substituents, illustratively representing methylamino, ethylamino, n-propylamino, isopropylamino, tert-butylamino, n-pentylamino, n-hexylamino, N,N-dimethylamino, N,N-diethylamino, N-ethyl-N-methylamino, N-methyl-N-n-propylamino, N-isopropyl-N-n-propylamino, N-t-butyl-N-methylamino, N-ethyl-N-n-pentylamino and N-n-hexyl-N-methylamino.

Alylaminocarbonyl represents an alkylaminocarbonyl radical having one or two (independently selected) alkyl substituents, illustratively representing methylaminocarbonyl, ethylaminocarbonyl, n-propylaminocarbonyl, isopropylaminocarbonyl, tert-butylaminocarbonyl, n-pentylaminocarbonyl, n-hexylaminocarbonyl, N,N-dimethylaminocarbonyl, N,N-diethylaminocarbonyl, N-ethyl-N-methylaminocarbonyl, N-methyl-N-n-propylaminocarbonyl, N-isopropyl-N-n-propylaminocarbonyl, N-t-butyl-N-methylaminocarbonyl, N-ethyl-N-n-pentylamino-carbonyl and N-n-hexyl-N-methyl-aminocarbonyl.

Alylaminosulfonyl represents an aminosulfonyl radical having one or two (independently selected) alkyl substitutents on the amino moiety, illustratively representing methylaminosulfonyl, ethylaminosulfonyl, n-propylaminosulfonyl, isopropylaminosulfonyl, tert-butylaminosulfonyl, n-pentylaminosulfonyl, n-hexyl-aminosulfonyl, N,N-dimethylaminosulfonyl, N,N-diethylaminosulfonyl, N-ethyl-N-methylaminosulfonyl, N-methyl-N-n-propylaminosulfonyl, N-isopropyl-N-n-propylaminosulfonyl, N-t-butyl-N-methylaminosulfonyl, N-ethyl-N-n-pentylaminosulfonyl and N-n-hexyl-N-methylaminosulfonyl.

Alkylsulfonylamino represents a sulfonylamino radical having an alkyl substitutent on the sulfonylamino moiety, illustratively representing methylsulfonylamino, ethylsulfonylamino, n-propylsulfonylamino, isopropylsulfonylamino, tert-butyl-sulfonylamino, n-pentylsulfonylamino and n-hexylsulfonylamino.

Aryl represents a mono- to tricyclic carbocyclic radical, which is aromatic at least in one ring and bound via an oxygen atom, having generally 6 to 14 carbon atoms, illustratively representing phenyl, naphthyl and phenanthrenyl.

Arylcarbonyl represents a carbonyl radical having an aryl substituent, illustratively and preferably represents phenylcarbonyl and naphthylcarbonyl.

Heteroaryl represents an mono- or bicyclic radical having 5 to 10 or 5 or 6 ring atoms and up to 5 or up to 4 hetero atoms selected from the group consisting of nitrogen, oxygen and sulfur, which is aromatic at least in one ring. It can be attached via a ring carbon atom or a ring nitrogen atom. If it represents a bicycle, wherein one ring is aromatic and the other one is not, it can be attached at either ring. Illustrative examples are thienyl, furyl, pyrrolyl, thiazolyl, oxazolyl, imidazolyl, pyridyl, pyrimidyl, pyridazinyl, indolyl, indazolyl, benzofuranyl, benzothiophenyl, quinolinyl and isoquinolinyl.

Halo or halogen represents fluorine, chlorine, bromine or iodine.

A * symbol next to a bond denotes the point of attachment in the molecule.

Throughout this document, for the sake of simplicity, the use of singular language is given preference over plural language, but is generally meant to include the plural language if not otherwise stated. E.g., the expression “A method of treating a disease in a patient, comprising administering to a patient an effective amount of a compound of claim 1” is meant to include the simultaneous treatment of more than one disease as well as the administration of more than one compound of claim 1.

In another embodiment, the present invention provides a process for preparing the compounds of formula (I), comprising reacting a compound of formula (II)

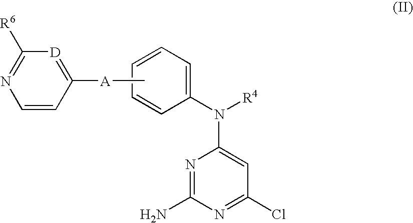

[A] with an agent of formula (IIIa)

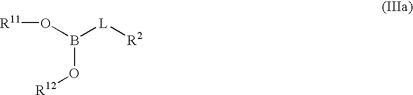

in which L and R² have the meaning indicated above, and R¹¹ and R¹² can be H or alkyl, or
[B] with an agent of formula (Ib)

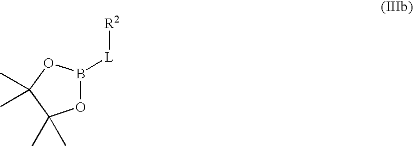

in which L and R² have the meaning indicated above, or
[C] with an agent of formula (IIc)

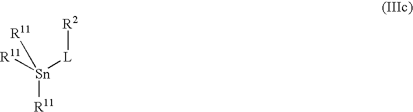

in which L, R² and R¹¹ have the meaning indicated above, in the presence of a suitable Pd catalyst, such as Pd₂(dba)₃ [tris(dibenzylideneacetone)-dipalladium(0)], Pd(PPh₃)₄ [tetrakis(triphenylphosphine)palladium(0)], or PdCl₂(dppf).CH₂Cl₂ {[1,1′-bis(diphenylphosphino)ferrocene]dichloropalladium(II) complex with dichloromethane}.

The compound of formula (II) can be prepared by condensation of a precursor of formula (VI)

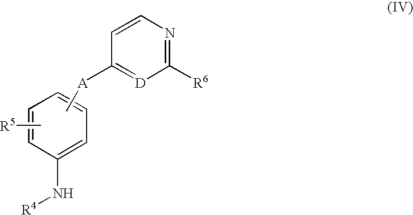

wherein A, D, and R⁴ to R⁶ have the meaning indicated above, with 2-amino-4,6-dichloropyrimidine.

Alternatively, compounds of formula (I) in which L represents ethanediyl are accessible by hydroboration of an alkene of formula (V)

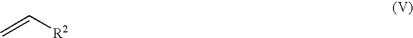

wherein R² has the meaning indicated above,
with a borane like 9-BBN and subsequent reaction with the intermediate of formula (II) in the presence of a Palladium catalyst such as Pd₂(dba)₃ [tris(dibenzylideneacetone)-dipalladium(0)], Pd(PPh₃)₄ [tetrakis(triphenylphosphine)palladium(0)], PdCl₂(dppf).CH₂Cl₂ ([1,1′-bis(diphenylphosphino)ferrocene]dichloropalladium(II) complex with dichloromethane}.

In case that L in formula (I) is ethendiyl, it can be converted into the corresponding single bond by catalytic hydrogenation. Also, residue R² in formula (I) might contain protecting groups that can be cleaved off.

In case that L in formula (I) represents ethynediyl, compounds of formula (I) can be prepared by reaction of a compound of formula (VI)

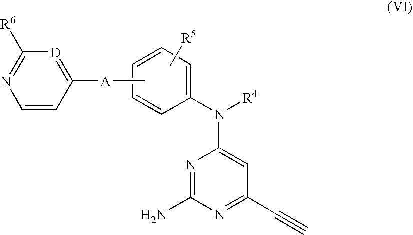

wherein D, and R⁴ to R⁶ have the meaning indicated above, with compounds of formula (VII)

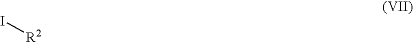

in which R² stands for an optionally substituted aromatic or heteroaromatic group in the presence of a palladium catalyst such as Pd(PPh₃)₂.Cl₂ and a copper salt such as copper(I)iodide.

Compounds of formula (VI) are available by reaction of precursor (IV) with a compound of formula (VI)

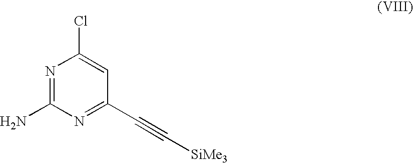

by a condensation reaction and subsequent removal of the trimethylsilyl group using a fluoride salt such as TBAF.

Compound (VIII) can be prepared by reacting commercially available 4-amino-2,6-dichlorpyrimidine with trimethylsilyl acetylene in the presence of a palladium catalyst such as bis(benzonitrile)dichloro palladium(I) and a copper salt such as copper(I)iodide.

It is also to be understood that starting materials are commercially available or readily prepared by standard methods well known in the art. Such methods include, but are not limited to the transformations listed herein.

If not mentioned otherwise, the reactions are usually carried out in inert organic solvents which do not change under the reaction conditions. These include ethers, such as diethyl ether, 1,4-dioxane or tetrahydrofuran, halogenated hydrocarbons, such as dichloromethane, trichloromethane, carbon tetrachloride, 1,2-dichloroethane, trichloroethane or tetrachloroethane, hydrocarbons, such as benzene, toluene, xylene, hexane, cyclohexane or mineral oil fractions, alcohols, such as methanol, ethanol or iso-propanol, nitromethane, dimethylformamide or acetonitrile. It is also possible to use mixtures of the solvents.

The reactions are generally carried out in a temperature range of from 0° C. to 150° C., preferably from 0° C. to 70° C. The reactions can be carried out under atmospheric, elevated or under reduced pressure (for example from 0.5 to 5 bar). In general, they are carried out under atmospheric pressure of air or inert gas, typically nitrogen.

The preparation of a compound of the present invention can be illustrated by means of the following synthetic methods:

### Method A:

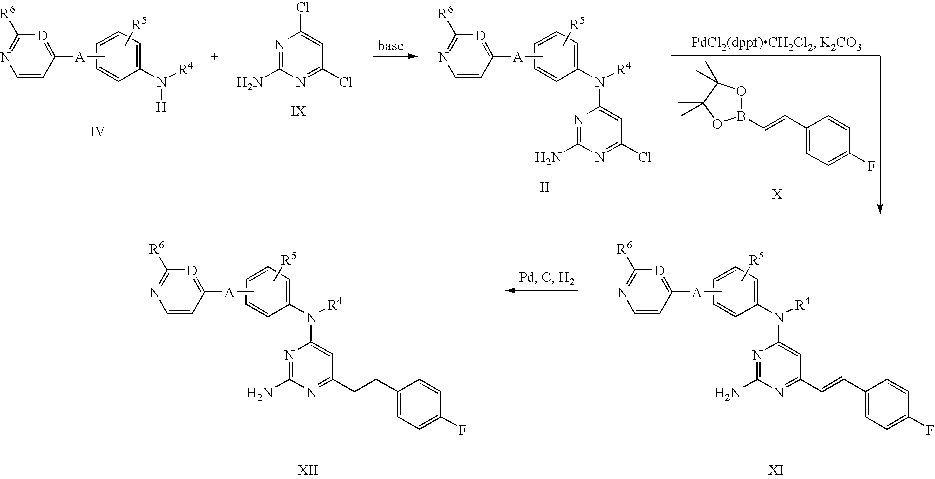

Thus a compound of formula (IV) can be condensed with the compound of formula (IX) in an inert solvent such as isopropanol and at elevated temperature in the presence or the absence of a base. Subsequently, intermediate of formula (ID) can be treated with a boronate such as of formula (X) in the presence of a palladium catalyst such as Pd₂(dba)₃ [tris(dibenzylideneacetone)-dipalladium(0)], Pd(PPh₃)₄ [tetrakis(triphenylphosphine)palladium(0)], or PdCl₂(dppf).CH₂Cl₂ {[1,1′-bis(diphenylphosphino)ferrocene]dichloropalladium(II) complex with dichloromethane} and a base such as potassium carbonate. The compound of formula (XI) can be converted into a compound of formula (XII) by catalytic hydrogenation in the presence of a palladium catalyst such as 10% palladium on charcoal.

### Method B:

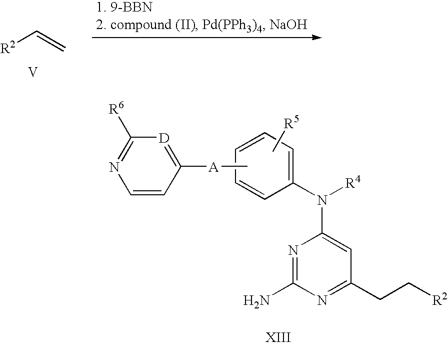

Alkene of formula (V) is hydroborated with an appropriate borane such as 9-BBN followed by Suzuki coupling employing intermediate of formula (II) in the presence of a palladium catalyst such as Pd₂(dba)₃ [tris(dibenzylideneacetone)-dipalladium(0)], Pd(PPh₃)₄ [tetrakis(triphenylphosphine)palladium(0)], or PdCl₂(dppf).CH₂Cl₂ {[1,1′-bis(diphenylphosphino)ferrocene]dichloropalladium(II) complex with dichloromethane} and a base such as potassium carbonate or sodium hydroxide.

Method C:

Treatment of 4-amino-2,6-dichloropyrimidine (IX) with trimethylsilylacetylene in the presence of a palladium catalyst such as bis(benzonitrile)dichloro palladium(II), a suitable ligand for example derived from [(tBu)₃PH]BF₄, a copperr(I) salt such as copper(I)iodide and a base such as diisopropylamine furnishes compound (VII), which is subsequently condensed with aniline (IV) to yield intermediate (XIV). From this, the trimethylsilyl group is cleaved off by treatment with a fluoride source such as TBAF and the resulting alkyne is reacted with a iodophenyl derivative such as 4-fluoroiodobenzene in the presence of a Palladium catalyst such as [bis(diphenylphosphino)]dichloropalladium(II) complex, a copper(I) salt such as copper(I)iodide and a base such as ethyl didopropylamine to yield compound (XV).

Many compounds of the present invention exhibit useful pharmacological and pharmacokinetic properties. They can therefore be useful for the treatment or prevention of disorders in humans and animals, especially hyperproliferative disorders such as cancer.

In another embodiment, the present invention provides a pharmaceutical composition comprising at least one compound according to the invention. In another embodiment, the present invention provides a pharmaceutical composition comprising at least one compound according to the invention together with one or more pharmacologically safe excipient or carrier substances. In a further embodiment, the present invention provides the use of said compound and composition for the treatment of a disease, as well as a method of treating a disease by administering to a patient a therapeutically effective amount of said compound or composition.

If used as active compounds, the compounds according to the invention are preferably isolated in more or less pure form, that is more or less free from residues from the synthetic procedure. The degree of purity can be determined by methods known to the chemist or pharmacist (see Remington's Pharmaceutical Sciences, 18th ed. 1990, Mack Publishing Group, Enolo). Preferably the compounds are greater than 99% pure (w/w), while purities of greater than 95%, 90% or 85% can be employed if necessary.

The present invention also relates to a method of using the compounds or compositions described herein for the treatment or prevention of, or in the manufacture of a medicament for treating or preventing, mammalian hyper-proliferative disorders. This method comprises administering to a patient (or a mammal) in need thereof, including a human, an amount of a compound, a pharmaceutically acceptable salt or ester thereof, or a composition of this invention, which is effective to treat or prevent the disorder.

Hyper-proliferative disorders include but are not limited to solid tumors, such as cancers of the breast, respiratory tract, brain, reproductive organs, digestive tract, urinary tract, eye, liver, skin, head and neck, thyroid, parathyroid and their distant metastases. Those disorders also include lymphomas, sarcomas, and leukemias.

The present invention also relates to a method for using the compounds of this invention as prophylactic or chemopreventive agents for prevention of the mammalian hyper-proliferative disorders described herein. This method comprises administering to a mammal in need thereof, including a human, an amount of a compound of this invention, or a pharmaceutically acceptable salt or ester thereof, which is effective to delay or diminish the onset of the disorder.

Examples of breast cancer include, but are not limited to invasive ductal carcinoma, invasive lobular carcinoma, ductal carcinoma in situ, and lobular carcinoma in situ.

Examples of cancers of the respiratory tract include, but are not limited to small-cell and non-small-cell lung carcinoma, as well as bronchial adenoma and pleuropulmonary blastoma.

Examples of brain cancers include, but are not limited to brain stem and hypophtalmic glioma, cerebellar and cerebral astrocytoma, medulloblastoma, ependymoma, as well as neuroectodermal and pineal tumor.

Tumors of the male reproductive organs include, but are not limited to prostate and testicular cancer. Tumors of the female reproductive organs include, but are not limited to endometrial, cervical, ovarian, vaginal, and vulvar cancer, as well as sarcoma of the uterus.

Tumors of the digestive tract include, but are not limited to anal, colon, colorectal, esophageal, gallbladder, gastric, pancreatic, rectal, small-intestine, and salivary gland cancers.

Tumors of the urinary tract include, but are not limited to bladder, penile, kidney, renal pelvis, ureter, and urethral cancers.

Eye cancers include, but are not limited to intraocular melanoma and retinoblastoma.

Examples of liver cancers include, but are not limited to hepatocellular carcinoma (liver cell carcinomas with or without fibrolamellar variant), cholangiocarcinoma (intrahepatic bile duct carcinoma), and mixed hepatocellular cholangiocarcinoma.

Skin cancers include, but are not limited to squamous cell carcinoma, Kaposi's sarcoma, malignant melanoma, Merkel cell skin cancer, and non-melanoma skin cancer.

Head-and-neck cancers include, but are not limited to laryngeal/hypopharyngeal/nasopharyngeal/oropharyngeal cancer, and lip and oral cavity cancer.

Lymphomas include, but are not limited to AIDS-related lymphoma, non-Hodgkin's lymphoma, cutaneous T-cell lymphoma, Hodgkin's disease, and lymphoma of the central nervous system.

Sarcomas include, but are not limited to sarcoma of the soft tissue, osteosarcoma, malignant fibrous histiocytoma, lymphosarcoma, and rhabdomyosarcoma.

Leukemias include, but are not limited to acute myeloid leukemia, acute lymphoblastic leukemia, chronic lymphocytic leukemia, chronic myelogenous leukemia, and hairy cell leukemia.

These disorders have been well characterized in humans, and also exist with a similar etiology in other mammals which can also be treated by the administration of the compounds and/or pharmaceutical compositions of the present invention.

In another embodiment, the present invention provides a medicament containing at least one compound according to the invention. In another embodiment, the present invention provides a medicament containing at least one compound according to the invention together with one or more pharmacologically safe excipient or carrier substances, for example hydroxypropylcellulose, and also their use for the above mentioned purposes.

The active component can act systemically and/or locally. For this purpose, it can be applied in a suitable manner, for example orally, parenterally, pulmonally, nasally, sublingually, lingually, buccally, rectally, transdermally, conjunctivally, otically or as an implant.

For these application routes, the active component can be administered in suitable application forms. An overview of application forms is given in Remington's Pharmaceutical Sciences, 18^{th} ed. 1990, Mack Publishing Group, Enolo.

Useful oral application forms include application forms which release the active component rapidly and/or in modified form, such as for example tablets (non-coated and coated tablets, for example with an enteric coating), capsules, sugar-coated tablets, granules, pellets, powders, emulsions, suspensions, solutions and aerosols. Such sustained-release pharmaceutical compositions are described in Part 8, Chapter 91 of Remington's Pharmaceutical Sciences, 18^{th} ed. 1990, Mack Publishing Group, Enolo.

Parenteral application can be carried out with avoidance of an absorption step (intravenously, intraarterially, intracardially, intraspinally or intralumbarly) or with inclusion of an absorption (intramuscularly, subcutaneously, intracutaneously, percutaneously or intraperitoneally). Useful parenteral application forms include injection and infusion preparations in the form of solutions, suspensions, emulsions, lyophilisates and sterile powders. Such parenteral pharmaceutical compositions are described in Part 8, Chapter 84 of Remington's Pharmaceutical Sciences, 18^{th} ed. 1990, Mack Publishing Group, Enolo.

In one embodiment, the invention relates to intravenous (i.v.) application of the active compound, e.g. as bolus injection (that is as single dose, e.g. per syringe), infusion over a short period of time (e.g. for up to one hour) or infusion over a long period of time (e.g. for more than one hour). The application can also be done by intermittent dosing. The applied volume can vary dependent on the conditions and usually is 0.5 to 30, or 1 to 20 ml for bolus injection, 25 to 500, or 50 to 250 ml for infusion over a short period of time and 50 to 1000, or 100 to 500 ml for infusion over a long period of time.

Such application forms have to be sterile and free of pyrogens. They can be based on aqueous solvents or mixtures of aqueous and organic solvents. Examples are ethanol, polyethyleneglycol (PEG) 300 or 400, aqueous solutions containing cyclodextrins or emulsifiers, such as lecithin, Pluronic F68®, Solutol HS15® or Cremophor®. Aqueous solutions are preferred.

For intravenous application the solutions are generally isotonic and euhydric, for example with a pH of 3 to 11, 6 to 8 or about 7.4.

Glass or plastic containers can be employed as packaging for i.v.-solutions, e.g. rubber seal vials. They can contain liquid volumes of 1 to 1000, or 5 to 50 ml. The solution can directly be withdrawn from the vial to be applied to the patient. For this purpose, it can be advantageous to provide the active compound in solid form (e.g. as lyophilisate) and dissolve by adding the solvent to the vial directly before administration.

Solutions for infusion can advantageously be packaged in containers made from glass or plastic, for example bottles or collapsible containers such as bags. They can contain liquid volumes of 1 to 1000, or 50 to 500 ml.

Forms suitable for other application routes include for example inhalatory pharmaceutical forms (including powder inhalers, nebulizers), nasal drops/solutions, sprays; tablets or capsules to be administered lingually, sublingually or buccally, suppositories, ear and eye preparations, vaginal capsules, aqueous suspensions (lotions, shake mixtures), lipophilic suspensions, ointments, creams, milk, pastes, dusting powders or implants.

The active components can be converted into said application forms in a manner known per se. This is carried out using inert non-toxic, pharmaceutically suitable excipients. These include inter alia carriers (for example microcrystalline cellulose), solvents (for example liquid polyethylene glycols), emulsifiers (for example sodium dodecyl sulphate), dispersing agents (for example polyvinylpyrrolidone), synthetic and natural biopolymers (for example albumin), stabilizers (for example antioxidants such as ascorbic acid), colorants (for example inorganic pigments such as iron oxides) or taste and/or odor corrigents. Exemplary application forms are given in part C of this application.

For human use, in the case of oral administration, it is recommended to administer doses of from 0.001 to 50 mg/kg, or from 0.01 to 20 mg/kg. In the case of parenteral administration such as, for example, intravenously or via mucous membranes nasally, buccally or inhalationally, it is recommended to use doses of 0.001 to 0.60 mg/kg, in particular 0.01 to 30 mg/kg.

In spite of this, it can be necessary in certain circumstances to depart from the amounts mentioned, namely as a function of body weight, application route, individual behaviour towards the active component, manner of preparation and time or interval at which application takes place. It can for instance be sufficient in some cases to use less than the aforementioned minimum amount, while in other cases the upper limit mentioned will have to be exceeded. In the case of the application of larger amounts, it can be advisable to divide them into a plurality of individual doses spread through the day.

The percentages in the tests and examples, which follows are, unless otherwise stated, by weight; parts are by weight. Solvent ratios, dilution ratios and concentrations reported for liquid/liquid solutions are each based on the volume.

### A. EXAMPLES

### Abbreviations and Acronyms

A comprehensive list of the abbreviations utilized by organic chemists of ordinary skill in the art appears in the first issue of each volume of the Journal of Organic Chemistry; this list is typically presented in a table entitled Standard List of Abbreviations. The abbreviations contained in said list, and all abbreviations utilized by organic chemists of ordinary skill in the art are hereby incorporated by reference.

For purposes of this invention, the chemical elements are identified in accordance with the Periodic Table of the Elements, CAS version, Handbook of Chemistry and Physics, 67th Ed., 1986-87.

More specifically, when the following abbreviations are used throughout this disclosure, they have the following meaning:
2× two times
3× three times
AlMe₃ trimethylaluminum
Boc t-butoxycarbonyl
n-BuLi butyllithium
t-BuOK potassium t-butoxide
calcd calculated
Celite® diatomaceous earth filtering agent, registered trademark of Celite Corp.
CD₃OD methanol-d₄
CHCl₃-d chloroform-d
d doublet
DBU 1,8-diazobicyclo[5.4.0]undec-7-ene
DCC dicyclohexylcarbodiimide
DEAD diethylazodicarboxylate
DIBAH diisobutylaluminum hydride
DIEA diisopropylethylamine
DMA dimethylacetamide
DMAP 4-dimethylaminopyridine
DME dimethoxyethane
DMF N,N-dimethylformamide
DMSO dimethylsulfoxide
DMSO-d₆ dimethylsulfoxide-d₆
EDCI 1-(3-dimethylaminopropyl)-3-ethylcarbodiide hydrochloride
EtSH ethanethiol
EtOAc ethyl acetate
EtOH ethanol
Et₃SiH triethylsilane
h hour(s)
HATU O-(7-azabenzotriazol-1-yl)-N,N,N′,N′-tetramethyluronium hexafluorophosphate
Hex hexanes
¹H NMR proton nuclear magnetic resonance
HOAc acetic acid
HPLC high performance liquid chromatography
LC-MS liquid chromatography/mass spectroscopy
LDA lithium diisopropylamide
LiHMDS lithium hexamethyldisilazide
m multiplet
m-CPBA 3-chloroperoxybenzoic acid
MeOH methanol
min minute(s)
Me₃SiI trimethylsilyl iodide
MS ES mass spectroscopy with electrospray
NaBH(OAc)₃ sodium triacetoxyborohydride
OMs O-methanesulfonyl (mesylate)
OTs O-p-toluenesulfononyl (tosyl)
OTf O-trifluoroacetyl (triflyl)
Pd/C palladium on carbon
Pd₂(dba)₃ tris(dibenzylideneacetone)dipalladium(0)
Pd(PPh₃)₄ tetrakis(triphenylphosphine)palladium(0)
PdCl₂(dppf).CH₂Cl₂ [1,1′-bis(diphenylphosphino)ferrocene]dichloropalladium(II) complex with dichloromethane
RT retention time
rt room temperature
Rf TLC Retention factor
s singlet
t triplet
TFA trifluoroacetic acid
THF tetrahydrofuran
TLC thin layer chromatography

### General Analytical Procedures

The structure of representative compounds of this invention were confirmed using the following procedures.

Electron impact mass spectra (EI-MS) were obtained with a Hewlett Packard 5989A mass spectrometer equipped with a Hewlett Packard 5890 Gas Chromatograph with a J & W DB-5 column (0.25 uM coating; 30 m×0.25 mm). The ion source was maintained at 250° C. and spectra were scanned from 50-800 amu at 2 sec per scan.

High pressure liquid chromatography-electrospray mass spectra (LC-MS) were obtained using either a:

(A) Hewlett-Packard 1100 HPLC equipped with a quaternary pump, a variable wavelength detector set at 254 nm, a YMC pro C-18 column (2×23 mm, 120 A), and a Finnigan LCQ ion trap mass spectrometer with electrospray ionization. Spectra were scanned from 120-1200 amu using a variable ion time according to the number of ions in the source. The eluents were A: 2% acetonitrile in water with 0.02% TFA and B: 2% water in acetonitrile with 0.018% TFA. Gradient elution from 10% B to 95% over 3.5 min at a flowrate of 1.0 mL/min is used with an initial hold of 0.5 min and a final hold at 95% B of 0.5 min. Total run time is 6.5 min.
or
(B) Gilson HPLC system equipped with two Gilson 306 pumps, a Gilson 215 Autosampler, a Gilson diode array detector, a YMC Pro C-18 column (2×23 mm, 120 A), and a Micromass LCZ single quadrupole mass spectrometer with z-spray electrospray ionization. Spectra were scanned from 120-800 amu over 1.5 seconds. ELSD (Evaporative Light Scattering Detector) data is also acquired as an analog channel. The eluents were either A: 2% acetonitrile in water with 0.02% TFA or B: 2% water in acetonitrile with 0.018% TFA. Gradient elution from 10% B to 90% over 3.5 min at a flowrate of 1.5 ml/min is used with an initial hold of 0.5 min and a final hold at 90% B of 0.5 min. Total run time is 4.8 min. An extra switching valve is used for column switching and regeneration.

Routine one-dimensional NMR spectroscopy is performed on 400 MHz Varian Mercury-plus spectrometers. The samples were dissolved in deuterated solvents obtained from Cambridge Isotope Labs, and transferred to 5 mm ID Wilmad NMR tubes. The spectra were acquired at 293 K. The chemical shifts were recorded on the ppm scale and were referenced to the appropriate solvent signals, such as 2.49 ppm for DMSO-d₆, 1.93 ppm for CD₃CN-d₃, 3.30 ppm for CD₃OD 5.32 ppm for CD₂Cl₂-d₂ and 7.26 ppm for CHCl₃-d for ¹H spectra.

### General HPLC Purification Method

Preparative reversed-phase HPLC chromatography was accomplished using a Gilson 215 system, typically using a YMC Pro-C18 AS-342 (150×20 mm I.D.) column. Typically, the mobile phase used was a mixture of (A) H₂O containing 0.1% TFA, and (B) acetonitrile. A typical gradient was:

| Time | | | Flow |
|---|---|---|---|
| [min] | A: % | B: % | [mL/min] |
| 0.50 | 90.0 | 10.0 | 1.0 |
| 11.00 | 0.0 | 100.0 | 1.0 |
| 14.00 | 0.0 | 100.0 | 1.0 |
| 15.02 | 100.0 | 0.0 | 1.0 |

### Intermediate 1A: 4-{4-[(2-amino-6-chloropyrimidin-4-yl)amino]phenoxy}pyridine-2-carbonitrile

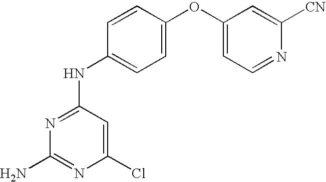

### Step 1: Preparation of 4-(4-aminophenoxy)pyridine-2-carbonitrile

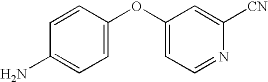

A three-neck, 3 L round bottomed flask fitted with a mechanical stirrer and a reflux condersor was charged with 4-aminophenol (41.35 g, 0.38 mol) and N,N-dimethylacetamide (500 mL). The resulting solution was degassed with bubbling nitrogen before potassium tert-butoxide was added portionwise (44.54 g, 0.40 mol). The solution became green at first, then became an off-white suspension, to which was added 4-chloropyridine-2-carbonitrile (50.00 g, 0.36 mol) in N,N-dimethylacetamide (300 mL) in one portion. The mixture turned brown within minutes and it was heated to 90° C. overnight. In the next morning, the mixture was cooled to rt and the solvent was removed under vacuum. The resulting residue was partitioned between water (1.5 L) and EtOAc (1.5 L). K₂CO₃ was added to adjust the pH to slightly basic and the layers were separated. The aqueous layer was extracted with EtOAc (1 L). The combined organic phase was dried over MgSO₄, filtered and concentrated. The resulting residue was dissolved in dichloromethane and absorbed onto a plug of silica gel (˜1 kg). It was then eluted with 25% to 75% EtOAc in hexanes to afford 4-(4-aminophenoxy)pyridine-2-carbonitrile (18.9 g, 25%): ¹H NMR (DMSO-d₆) δ ppm 8.48 (d, 1H), 7.51 (d, 1H), 7.04 (dd, 1H), 6.83 (dd, 2H), 6.60 (dd, 2H), 5.18 (s, 2H); MS ES 212 (M+H), RT 0.97 min.

### Step 2: Preparation of the Title Compound

A three-neck, 3 L round bottomed flask fitted with a mechanical stirrer and a reflux condesror was charged with 4-(4-aminophenoxy)pyridine-2-carbonitrile (70.00 g, 0.33 mol), 4,6-dichloropyrimidin-2-amine (54.35 g, 0.33 mol), water (2.5 L), and 2-propanol (500 mL). The suspension was heated to 91° C. for 4 hours before it was cooled to rt overnight. The reaction mixture was filtered and the solid collected was washed with EtOH, ether and hexanes. The solid was dried by air suction for 45 min to give 4-{4-[(2-amino-6-chloropyrimidin-4-yl)amino]phenoxy}pyridine-2-carbonitrile (84.1 g, 75%): ¹H NMR (DMSO-d₆) δ ppm 9.45 (s, 1H), 8.55 (d, 1H), 7.80 (d, 2H), 7.64 (d, 1H), 7.12-7.15 (m, 3H), 6.76 (s, 2H), 6.00 (s, 1H), 3.34 (s, 2H); MS ES 339 (M+H), RT 2.49 min.

### Intermediate 1B: 6-chloro-N⁴-(4-{[2-(trifluoromethyl)pyridin-4-yl]oxy}phenyl)pyrimidine-2,4-diamine

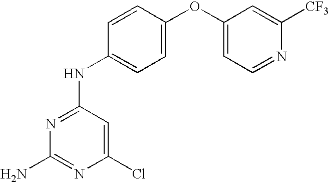

It was prepared in a two-step sequence similar to what was described for intermediate 1A: ¹H NMR (DMSO-d₆) δ ppm 9.46 (s, 1H), 8.59 (d, 1H), 7.81 (d, 2H), 7.37 (d, 1H), 7.17 (d, 2H), 7.11 (dd, 1H), 6.78 (s, 2H), 6.00 (s, 1H). MS ES 382 (M+H), calcd 382 RT 2.93 min.

### Intermediate 1C: 6-chloro-N⁴-{4-[(2-methylpyridin-4-yl)oxy]phenyl}pyrimidine-2,4-diamine

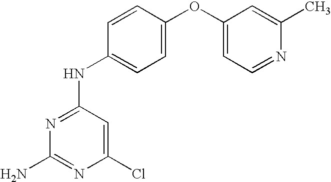

It was prepared in a two-step sequence similar to what was described for intermediate 1A: ¹H NMR (DMSO-d₆) [symbol] 9.40 (s, 1H), 8.27 (d, 1H), 7.76 (d, 2H), 7.06 (d, 2H), 6.75 (brs, 2H), 6.72 (d, 1H), 6.66 (s, 1H), 5.98 (s, 1H); MS ES 328 (M+H)⁺, calcd 328, RT=1.45 min.

### Intermediate 1D: 4-{3-[(2-amino-6-chloropyrimidin-4-yl)amino]phenoxy}pyridine-2-carbonitrile

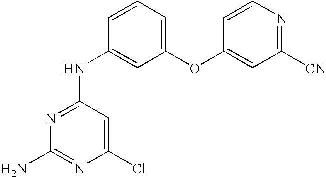

It was prepared in a two-step sequence similar to what was described for intermediate 1A: ¹H NMR (DMSO-d₆) [symbol] 9.52 (s, 1H), 8.57 (d, 1H), 7.72 (dd, 1H), 7.69 (d, 1H), 7.53 (dd, 1H), 7.38 (dd, 1H), 7.18 (dd, 1H), 6.77-6.80 (m, 3H), 6.01 (s, 1H); MS ES 339 (M+H)⁺, calcd 339, RT=2.65 min.

### Intermediate 2A: 4-{3-[(2-amino-6-chloropyrimidin-4-yl)amino]phenoxy}pyridine-2-carboxamide

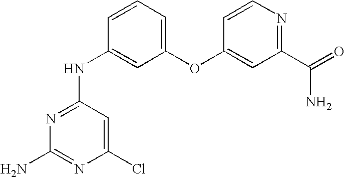

To a 100 mL round bottomed flask was charged with 4-{3-[(2-amino-6-chloropyrimidin-4-yl)amino]phenoxy}pyridine-2-carbonitrile (intermediate 1D, 5.00 g, 14.8 mmol) and concentrated sulfuric acid (40 mL). The mixture was heated to 70° C. for 2 hours before it was cooled to rt. It was then slowly poured into NaHCO₃ and ice water mixture before EtOAc was added with stirring. The organic layer was separated, dried over MgSO4, and filtered. The filtrate was concentrated in vacuo to afford 4-{3-[(2-amino-6-chloropyrimidin-4-yl)amino]phenoxy}pyridine-2-carboxamide as a colorless powder (4.50 g, 85%): [symbol] 9.51 (s, 1H), 8.49 (d, 1H), 8.13 (d, 1H), 7.72 (d, 1H), 7.66-7.68 (m, 1H), 7.54 (dd, 1H), 7.43 (d, 1H), 7.38 (dd, 1H), 7.18 (dd, 1H), 6.77-6.81 (m, 3H), 6.00 (s, 1H); MS ES 357 (M+H)⁺, calcd 357, RT=2.32 min.

### Example 1

### Preparation of 6-(1-ethoxyvinyl)-N⁴-(4-{[2-(trifluoromethyl)pyridin-4-yl]oxy}phenyl)pyrimidine-2,4-diamine

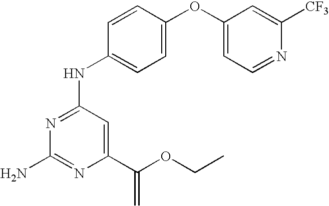

6-chloro-N⁴-(4-{[2-(trifluoromethyl)pyridin-4-yl]oxy}phenyl)pyrimnidine-2,4-diamine (100 mg, 0.26 mmol), Pd(PPh₃)₄ (15.2 mg, 0.01 mmol), K₂CO₃ (43.5 mg, 0.31 mmol), toluene (3 mL) and DMA (1 mL) were placed in a 8 ml microwave vial. The mixture was degassed for a few minutes and then tributyl(1-ethoxyl)tin (113.5 mg, 0.31 mmol) was added and the reaction mixture was heated in microwave reactor (Emrys optimizer by Personal Chemistry) for 15 minutes at 180° C. The mixture was cooled and filtered over celite and the solvent was evaporated. The resulting mixture was purified with silica gel chromatography (3:2 hexane:EtOAc, then EtOAc) to provide the title compounds as a slightly yellow oil. ¹H NMR (DMSO-d₆) [symbol] 9.42 (s, 1H), 8.58 (m, 1H), 7.90 (m, 2H), 7.30 (m, 1H), 7.15 (m, 3H), 6.25 (s, 1H), 5.21 (s, 2H), 5.30 (s, 1H), 4.25 (s, 1H), 8.32 (q, 2H), 1.35 (t, 3H); MS ES 417.9 (M+H)⁺, calcd 418.1.

### Example 2

### Preparation of 1-{2-amino-6-[(4-{[2-(trifluoromethyl)pyridin-4-yl]ox-}phenyl)amino]pyrimidin-4-yl}ethanol

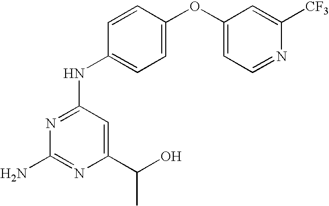

### Step 1: Preparation of 1-{2-amino-6-[(4-{[2-(trifluoromethyl)pyridin-4-yl]oxy}phenyl)amino]pyrimidin-4-yl}ethanone

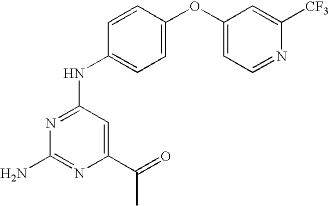

6-(1-ethoxyvinyl)-N⁴-(4-{[2-(trifluoromethyl)pyridin-4-yl]oxy}phenyl)pyrimidine-2,4-diamine (example 1) was dissolved in 5 ml THF and 3 ml 2 N aqueous HCl was added. The reaction mixture was stirredat rt overnight. Most of THF was evaporated and the mixture was basified with saturated NaHCO₃, extracted with EtOAc, washed with water, brine, dried and filtered. The filtrate was concentrated to provide 1-{2-amino-6-[(4-{[2-(trifluoromethyl)pyridin-4-yl]oxy}phenyl)amino]pyrimidin-4-yl}ethanone as a slightly yellow solid. ¹H NMR (CD₃OD) [symbol] 8.55 (m, 1H), 7.85 (m, 2H), 7.31 (s, 1H), 7.15 (m, 2H), 7.12 (m, 1H), 6.62 (s, 1H), 2.55 (s, 3H); MS ES 389.9 (M+H)⁺, calcd 389.1.

### Step 2: Preparation of Title Compound

1-{2-amino-6-[(4-{[2-(trifluoromethyl)pyridin-4-yl]oxy}phenyl)amino]-pyrimidin-4-yl}ethanone (30 mg, 0.08 mmol), propylamine (9.11 mg, 0.15 mmol), NaBH₃CN (9.7 mg, 0.15 mmol) were dissolved in 1 mL MeOH and 1 mL THF. The reaction mixture was stirred at rt overnight. The solvent was evaporated and the resulting mixture was purified by preparative HPLC to provide 6-[1-(propylamino)ethyl]-N⁴-(4-{[2-(trifluoromethyl)pyridin-4-yl]oxy}phenyl)pyrimidine-2,4-diamine (8.5 mg, 25% yield) and the title compound (15 mg, 48% yield). ¹H NMR (CD₃OD) [symbol] 8.55 (m, 1H), 7.90 (m, 2H), 7.35 (s, 1H), 7.25 (m, 2H), 7.15 (m, 1H), 6.25 (s, 1H), 4.75 (q, 1H), 1.55 (d, 3H); MS ES 391.9 (M+H)⁺, calcd 391.3.

### Example 3

### Preparation of 4-[4-({2-amino-6-[(E)-2-(4-fluorophenyl)vinyl]pyrimidin-4-yl}amino)phenoxy]pyridine-2-carbonitrile

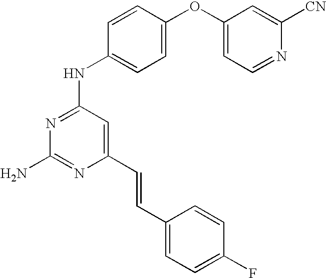

To a mixture of 1 equivalent of 4-{4-[(2-amino-6-chloropyrimidin-4-yl)amino]phenoxy}pyridine-2-carbonitrile (100 mg,), 2 equivalents of [(E)-2-(4-fluorophenyl)vinyl]boronic acid, and 0.06 equivalent of PdCl₂(dppf) CH₂Cl₂ complex in 2.3 mL anhydrous N,N-dimethylacetamide in a 8 mL microwave reaction vessel was added 3.1 equivalent of 2M K₂CO₃ aqueous solution. After the resulting mixture was degassed for 10 min using N₂, the vial was sealed and heated at 140° C. for 20 min in a microwave reactor (Emrys optimizer by Personal Chemistry). The reaction mixture was filtered, and the filtrate was concentrated and purified by prep-HPLC eluting with 15% to 85% acetonitrile using a Phenomenex Luna 5μ C18 150×30 mm column to provide the final product.

By using the appropriate starting materials, the method described for Example 3, was utilized for the preparation of Examples 4.

### Example 5

### 6-[(E)-2-(4-fluorophenyl)vinyl]-N⁴-(4-{[2-(trifluoromethyl)pyridin-4-yl]oxy}phenyl)pyrimidine-2,4-diamine

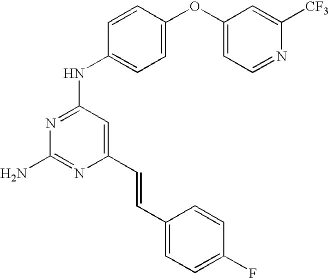

To a mixture of 1 equivalent of 6-chloro-N4-(4-{[2-(trifluoromethyl)pyridin-4-yl]oxy}phenyl)pyrimidine-2,4-diamine (100 mg), 2 equivalent of [(E)-2-(4-fluorophenyl)vinyl]boronic acid, and 0.06 equivalent of PdCl₂(dppf) CH₂Cl₂ complex in 2.3 mL anhydrous N,N-dimethylacetamide in a 5 mL microwave reaction vessel was added 3.1 equivalent of 2 M K₂CO₃ aqueous solution. After the resulting mixture was degassed for 10 min using N₂, the vial was sealed and heated at 150° C. for 20 min in a microwave reactor (Emrys optimizer by Personal Chemistry). The reaction mixture was filtered, and the filtrate was concentrated and purified by prep-HPLC eluting with 15% to 85% acetonitrile using a Phenomenex Luna 5μ C18 150×30 mm column to provide the final product.

By using the appropriate starting materials, the method described for Example 4, was utilized for the preparation of Examples 6-7.

### Example 8

### (3E)-4-[2-amino-6-[(4-{[2-(trifluoromethyl)pyridin-4-yl]oxy}phenyl)amino]pyrimidin-4-yl]but-3-en-1-ol

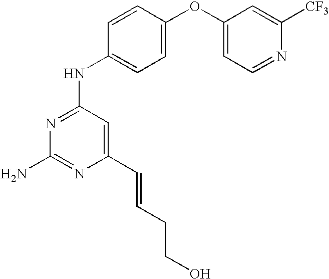

### Step 1: Preparation of 6-((1E)-4-{[tert-butyl(dimethyl)silyl]oxy}but-1-en-1-yl)-N⁴-(4-{[2-(trifluoromethyl)pyridin-4-yl]oxy}phenyl)pyrimidine-2,4-diamine

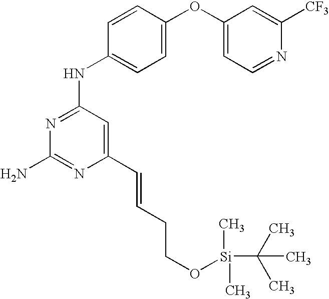

By using commercially available trans-1-buten-1-yl-(4-ter-butyldimethylsilyloxy-4′,4′,5′,5′-tetramethyl-(1′,3′,2′)-dioxaborolane and 6-chloro-N⁴-(4-{[2-(trifluoromethyl)pyridin-4-yl]oxy}phenyl)pyrimidine-2,4-diamine, the above compound was prepared in similar manner as described in example 3: ¹H NMR (DMSO-d₆) δ 8.48 (d, 1H), 7.42 (m, 2H), 7.15 (d, 1H), 7.01 (m, 2H), 6.91 (m, 1H), 6.76 (m, 1H), 6.49 (s, 1H), 6.15 (m, 2H), 5.92 (s, 1H), 4.82 (br s, 2H), 3.67 (dd, 2H), 2.39 (m, 2H), 0.83 (s, 9H), 0.00 (s, 6H).

### Step 2: Preparation of the Title Compound

To a solution of 6-((1E)-4-{[tert-butyl(dimethyl)silyl]oxy}but-1-en-1-yl)-N⁴-(4-{[2-(trifluoromethyl)pyridin-4-yl]oxy}phenyl)pyrimidine-2,4-diamine (128 mg, 0.24 mmol) in 2.4 mL anhydrous methylene chloride was added 0.5 mL TFA the solution was left stirring for 2 hours at rt before it was diluted with EtOAc and 5% aqueous NaHCO₃. The two layers were separated and the aqueous layer was extracted with EtOAc. The combined organic layers were washed with brine and dried with Na₂SO₄, filtered and concentrated. The resulting residue was triturated with 50% EtOAc/hexanes to obtain 99 mg product as a white solid. ¹H NMR (CD₃OD) δ 8.4 (d, 1H), 7.83 (d, 2H), 7.32 (d, 1H), 7.17 (m, 2H), 7.12 (m, 1H), 6.75 (m, 1H), 6.31 (d, 1H), 6.15 (s, 1H), 3.72 (t, 2H), 2.51 (q, 2H); MS ES 418 (M+H)⁺ calcd 418, RT=2.05 min.

### Example 14

### 6-(3-aminopropyl)-N⁴-(4-{[2-(trifluoromethyl)pyridin-4-yl]oxy}phenyl)pyrimidine-2,4-diamine hydrochloride

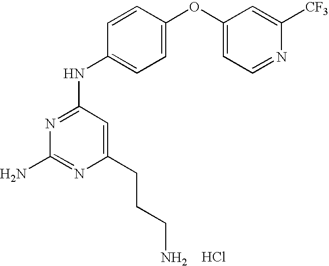

### Step 1: Preparation of tert-butyl (3-{2-amino-6-[(4-{[2-(trifluoromethyl)pyridin-4-yl]oxy}phenyl)amino]pyrimidin-4-yl}propyl)carbamate

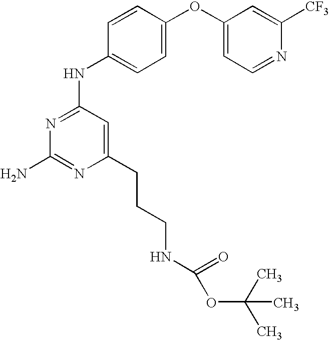

To a solution of t-Butyl N-allylcarbamate (75 mg, 0.48 mmol) in 1.5 mL anhydrous THF under N₂ was added 9-BBN (0.5 M in THF, 0.71 mmol) and the resulting solution was stirred for 3 hours at rt. To the above mixture was added 6-chloro-1-(4-{[2-(trifluoromethyl)-pyridin-4-yl]oxy}phenyl)pyrimidine-2,4-diamine (150 mg, 0.39 mmol), 1 mL THF, Pd(PPh₃)₄ (20 mg, 0.017 mmol), and NaOH (47 mg in 0.5 mL of water, 1.18 mmol) and the resulting mixture was heated to 70° C. overnight. In the morning, the reaction mixture was cooled to rt and EtOAc and water were added and the mixture was filtered through celite. The layers were separated and the aqueous layer was extracted with EtOAc. The combined organic layers were washed with brine and dried with Na₂SO₄, filtered and concentrated. The resulting residue was purified on Biotage column by using 25-100% EtOAc/hexanes and to give 78 mg (39%) of the desired compound. ¹H NMR (CD₂Cl₂) δ 8.55 (d, 1H), 7.53 (m, 2H), 7.25 (d, 1H), 7.12 (m, 2H), 7.03 (m, 1H), 6.75 (bs, 1H), 6.01 (s, 1H), 5.11 (bs, 1H), 5.0 (bs, 2H), 3.15 (m, 2H), 2.53 (t, 2H), 1.85 (m, 2H), 1.21 (s, 9H); MS ES 505(M+H)⁺ calcd 505, RT=2.69 min.

### Step 2: Preparation of the Title Compound

To a solution of tert-butyl (3-{2-amino-6-[(4-{[2-(trifluoromethyl)pyridin-4-yl]oxy}phenyl)amino]pyrimidin-4-yl}propyl)carbamate (50 mg, 0.10 mmol) in 0.4 mL anhydrous MeOH was added 4M HCl in dioxane (0.37 mL, 1.49 mmol) and the solution was stirred for 1 h at rt. The resulting solution was concentrated under vacuum to give 33 mg of the title compound as a tan solid. ¹H NMR (CD₂Cl₂) δ 12.92 (bs, 1H), 10.98 (bs, 1H), 8.61 (d, 1H), 7.96 (m, 6H), 7.39 (m, 1H), 7.26 (m, 2H), 7.13 (m, 1H), 6.28 (s, 1H), 2.85 (m, 2H), 2.67 (m, 2H), 1.93 (m, 2H)); MS ES 405(M+H)⁺ calcd 405, RT=2.17 min.

### Example 15

### 4-{2-amino-6-[(4-{[2-(trifluoromethyl)pyridin-4-yl]oxy}phenyl)amino]pyrimidin-4-yl}butan-1-ol

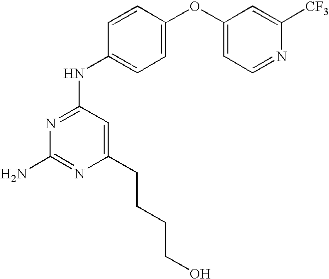

In a flask filled with N₂ was placed 10%/wt Palladium on activated carbon (6 mg) and (3E)-4-{2-amino-6-[(4-{[2-(trifluoromethyl)pyridin-4-yl]oxy}phenyl)amino]pyrimidin-4-yl}but-3-en-1-ol (example 8, 59 mg, 0.14 mmol, in 1 mL of EtOAc). The flask was purged with hydrogen from a balloon and stirred for 3 hours. The mixture was filtered to remove catalyst and the filtrate was concentrated to give 40 mg (67%) of the desired compound as a gray solid. ¹H NMR (DMSO-d₆) δ 9.14 (s, 1H), 8.58 (d, 1H), 7.83 (m, 2H), 7.35 (d, 1H), 7.11 (m, 3H), 6.16 (s, 2H), 5.86 (s, 1H), 4.37 (t, 1H), 3.39 (m, 2H), 2.34 (t, 2H), 1.6 (m, 2H), 1.46 (m, 2H); MS ES 420(M+H)⁺ calcd 420, RT=2.24 min.

### Example 16

### 4-{2-amino-6-[(4-{[2-(trifluoromethyl)pyridin-4-yl]oxy}phenyl)amino]pyrimidin-4-yl}propan-1-ol

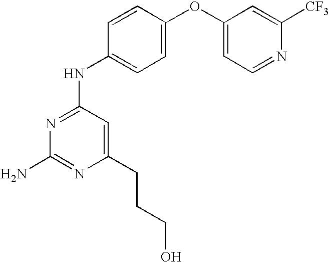

### Step 1: Preparation of 6-(3-{[tert-butyl(dimethyl)silyl]oxy}propyl)-N⁴-(4-{[2-(trifluoromethyl)pyridin-4-yl]oxy}phenyl)pyrimidine-2,4-diamine

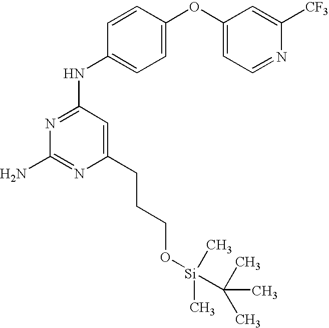

By using (allyloxy)(tert-butyl)dimethylsilane and 6-chloro-N⁴-(4-{[2-(trifluoromethyl)-pyridin-4-yl]oxy}phenyl)pyrimidine-2,4-diamine, the above compound was prepared in a similar manner as described in step 1 of Example 14: ¹H NMR (CD₂Cl₂) δ 8.47 (d, 1H), 7.48 (m, 2H), 7.17 (d, 1H), 7.03 (m, 2H), 6.94 (m, 1H), 6.61 (s, 1H), 5.92 (s, 1H), 5.20 (br s, 2H), 3.59 (t, 2H), 2.48 (m, 2H), 1.81 (m, 2H), 0.84 (s, 9H), 0.00 (s, 6H); MS ES 520 (M+H)⁺ calcd 520, RT=3.20.

### Step 2: Preparation of the title compound

By starting from 6-(3-{[tert-butyl(dimethyl)silyl]oxy}propyl)-N⁴-(4-{[2-(trifluoromethyl)pyridin-4-yl]oxy}phenyl)pyrimidine-2,4-diamine, the title compound was prepared in a similar manner as described in step 2 of Example 8: ¹H NMR (DMSO-d₆) δ 8.61 (d, 1H), 7.87 (s, 2H), 7.39 (s, 1H), 7.26 (m, 2H), 7.14 (m, 1H), 6.11 (s, 1H), 3.46 (t, 2H), 2.58 (m, 2H), 1.73 (m, 2H); MS ES 406 (M+H)⁺ calcd 406, RT=2.32.

### Example 17

### 4-{2-amino-6-[(4-{[2-(trifluoromethyl)pyridin-4-yl]oxy}phenyl)amino]pyrimidin-4-yl}butane-1,2-diol

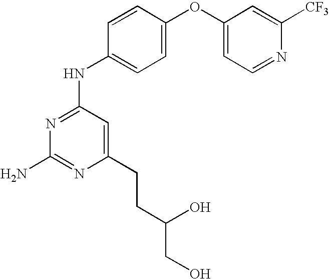

### Step 1: Preparation of 2,2,3,3,8,8,9,9-octamethyl-5-vinyl-4,7-dioxa-3,8-disiladecane

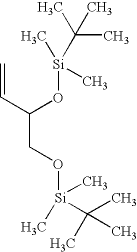

To a round bottomed flask was charged with 80 mL THF, imidazole (3.09 g, 45.4 mmo), 1 eq R,S-3-butene-1,2-diol (1.0 g, 11.4 mmol), and tert-butyldimethylsilyl chloride (6.84 g, 45.4 mmol). The resulting mixture was stirred at rt for 72 hours before it was removed under vacuum. Ether was added and the mixture was filtered. The filtrate was concentrated and the resulting residue was purified by silica gel column by using 0-50% EtOAc/hexanes to yield 2.1 g (58%) of the desired product as a clear oil. ¹H NMR (CD₂Cl₂) δ 5.80 (m, 1H), 5.22-5.17 (m, 1H), 5.05-5.0 (m, 1H), 4.09 (m, 1H), 3.43 (m, 2H), 0.84 (m, 18H), 0.0 (m, 12H).

### Step 2: Preparation of 6-(3,4-bis{[tert-butyl(dimethyl)silyl]oxy}butyl)-N⁴-(4-{[2-(trifluoromethyl)pyridin-4-yl]oxy}phenyl)pyrimidine-2,4-diamine

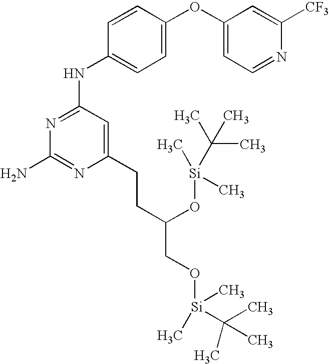

By using 2,2,3,3,8,8,9,9-octamethyl-5-vinyl-4,7-dioxa-3,8-disiladecane and 6-chloro-N⁴-(4-{[2-(trifluoromethyl)-pyridin-4-yl]oxy}phenyl)pyrimidine-2,4-diamine, the above compound was prepared in a similar manner as described in step 1 of Example 14: ¹H NMR (CD₂Cl₂) δ 8.46 (d, 1H), 7.46 (m, 2H), 7.16 (d, 1H), 7.02 (m, 2H), 6.93 (m, 1H), 6.51 (s, 1H), 5.89 (s, 1H), 4.84 (s, 2H), 3.66 (m, 1H), 3.37-3.53 (m, 2H), 2.36-2.57 (m, 2H), 1.86 (m, 1H), 1.65 (m, 1H), 0.84 (m, 18H), 0.01 (m, 12H); MS ES 664 (M+H)⁺ calcd 664, RT=3.80.

### Step 3: Preparation of the title compound

By starting from 6-(3,4-bis{[tert-butyl(dimethyl)silyl]oxy}butyl)-N⁴-(4-{[2-(trifluoromethyl)pyridin-4-yl]oxy}phenyl)pyrimidine-2,4-diamine, the title compound was prepared in a similar manner as described in step 2 of Example 8: ¹H NMR (DMSO-d₆) δ 8.61 (d, 1H), 7.89 (m, 2H), 7.39 (m, 1H), 7.25 (d, 2H), 7.14 (m, 1H), 6.15 (s, 1H), 4.62 (s, 1H), 3.45 (m, 1H), 3.26 (m, 1H), 2.66 (m, 1H), 2.56 (m, 1H), 1.92 (m, 1H), 1.55 (m, 1H); MS ES 436 (M+H)⁺ calcd 436, RT=2.20.

### Example 22

### 6-[(4-fluorophenyl)ethynyl]-N⁴-(4-{[2-(trifluoromethyl)pyridin-4-yl]oxy}phenyl)pyrimidine-2,4-diamine

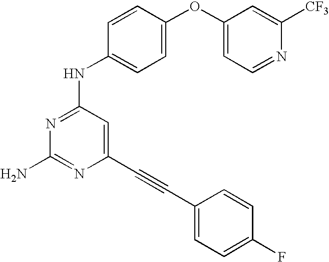

### Step 1: Preparation of 4-{[tert-butyl(dimethyl)silyl]ethynyl}-6-chloropyrimidin-2-amine

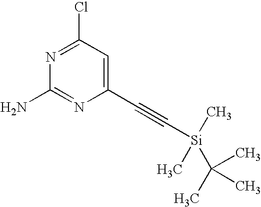

Under argon, 1-amino-2,6-dichloro pyrimidine (5.00 g, 30.5 mmol), bis(benzonitrile)dichloro palladium(II) (351 mg, 0.91 mmol), [(tBu)₃PH]BF₄ (575 mg, 1.98 mmol) and copper(I)iodide (116 mg, 0.61 mmol) were combined. 1,4-dioxane (40 ml), diisopropylamine (5.13 ml, 36.6 mmol) and trimethylsilyl acetylene (5.17 ml, 36.6 ml) were added and the mixture was heated to 40° C. for 48 h. The crude material was passed through a silica pad eluting with dichlor methane and concentrated in vacuo to result in brown glass that was used for the next step without purification.

### Step 2: Preparation of 6-{[tert-butyl(dimethyl)silyl]ethynyl}-N⁴-(4-{[2-(trifluoromethyl)pyridin-4-yl]oxy}phenyl)pyrimidine-2,4-diamine

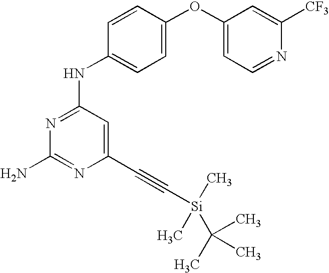

The crude product from step 1 (1.53 g, 6.7 mmol) and 4-{[2-(trifluoromethyl)pyridin-4-yl]oxy} aniline (1.03 g, 4.1 mmol) (prepared in similar manner as described in step 1 of intermediate 1A) were dissolved in isopropanol (10 ml) and heated to 50° C. for 16 h. The solvent was removed in vacuo to result in a dark yellow solid. The solid was treated with a mixture to 30 ml dichloromethane and 5 ml triethylamineAftyer concentration in vacuo the residue was purified by column chromatography (silica, dichloro methane:isopropanol 98:2 to result in 1.57 g (86%) of the intermediate.

### Step 3: preparation of 6-ethynyl-N⁴-(4-{[2-(trifluoromethyl)pyridin-4-yl]oxy}phenyl)pyrimidine-2,4-diamine

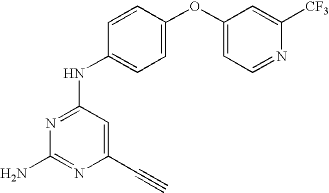

The product from step 2 (3.46 g, 7.8 mmol) was dissolved in wet THF (30 ml) and a solution of tetrabutyl ammonium fluoride (3.06 g, 11.7 mmol) in THF (10 ml) was added. The mixture was stirred for 1 h at room temperature. After concentration in vacuo, the residue was purified by silica gel chromatography (ethylacetate:hexane 4:1) to yield the desired intermediate 6-ethynyl-N⁴-(4-{[2-(trifluoromethyl)pyridin-4-yl]oxy}phenyl)pyrimidine-2,4-diamine (2.73 g, 94%). ¹H NMR (DMSO-d₆) δ 9.38 (s, 1H), 8.59 (d, 1H), 7.84 (m, 2H), 7.36 (d, 1H), 7.16 (m, 2H), 7.11 (m, 1H), 6.47 (s, 2H), 6.3 (s, 1H), 4.23 (s, 1H).

### Step 4: Preparation of the title compound

In evacuated vial filled with N₂ was placed 6-ethynyl-N⁴-(4-{[2-(trifluoromethyl)pyridin-4-yl]oxy}phenyl)pyrimidine-2,4-diamine (150 mg, 0.40 mmol), Pd(PPh₃)₂.Cl₂ (15 mg, 0.020 mmol), CuI (4 mg, 0.020 mmol), 4-Fluoroiodobenzene (0.44 mmol) and 4 mL anhydrous DMF. To the above mixture was added N,N-Diisopropylethylamine (0.21 mL, 1.2 mmol) the reaction mixture was heated to 80° C. overnight. In the morning, the mixture was cooled to rt and concentrated under vacuum. The resulting residue was purified by Biotage 25M column eluting with EtOAc/hexanes (1/1) to give 116 mg of the title compound (62%). ¹H NMR (DMSO-d₆) δ 9.40 (s, 1H), 8.59 (d, 1H), 7.85 (m, 2H), 7.62 (m, 2H), 7.37 (d, 1H), 7.28 (m, 2H), 7.16 (m, 2H), 7.12 (m, 1H), 6.49 (s, 1H), 6.21 (s, 2H); MS ES 466 (M+H)⁺ calcd 466, RT=2.89.

By using the method described, and by substituting appropriate starting materials, examples 9, 10, 11, 20, 21, 23, 24, 25, 26, 33 were also prepared.

### Example 29

### 6-[2-(4-fluorophenyl)ethyl]-N⁴-(4-{[2-(trifluoromethyl)pyridin-4-yl]oxy}phenyl)pyrimidine-2,4-diamine

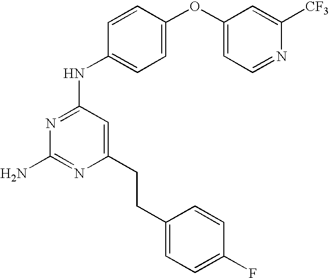

In a round bottomed flask filled with N₂ was placed 10%/wt Palladium on activated carbon (9 mg), 6-[(4-fluorophenyl)ethynyl]-N⁴-(4-{[2-(trifluoromethyl)pyridin-4-yl]oxy}phenyl)pyrimidine-2,4-diamine (example 22, 90 mg, 0.19 mmol) and 2 mL of EtOAc/MeOH (10:1). The flask was purged with hydrogen from a balloon and stirred overnight. In the morning, the reaction mixture was filtered and concentrated under vacuum to give 77 mg (85%) of the title compound as a solid: ¹H NMR (DMSO-d₆) δ 9.14 (s, 1H), 8.58 (d, 1H), 7.82 (m, 2H), 7.35 (d, 1H), 7.22 (m, 2H), 7.14-7.04 (m, 5H), 6.21 (s, 2H), 5.84 (s, 1H), 2.89 (m, 2H), 2.64 (m, 2H); MS ES 470 (M+H)⁺ calcd 470, RT=2.69 min.

By using the method described, and by substituting appropriate starting materials, examples 12, 13, 18, 27, 28, 30, 31, 32, 34, 37, and 38 were also prepared.

### Example 35

### 3-(2-{2-amino-6-[(4-{[2-(trifluoromethyl)pyridin-4-yl]oxy}phenyl)-amino]pyrimidin-4-yl}ethyl)phenol

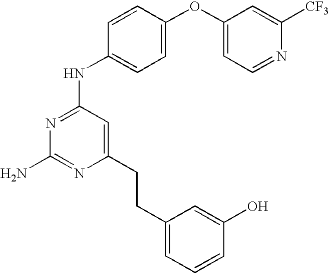

In a round bottomed flask under N₂, 6-[2-(3-methoxyphenyl)ethyl]-N4-(4-{[2-(trifluoromethyl)pyridin-4-yl]oxy}phenyl)pyrimidine-2,4-diamine (example 31, 38 mg, 0.079 mmol) was dissolved in 0.8 mL anhydrous methylene chloride. To the above mixture, boron tribromide-methyl sulfide complex (0.79 mL, 0.79 mmol) was added dropwise and the mixture was stirred overnight. In the morning, water (2 mL) was added followed by aqueous saturated NaHCO₃ until bubbling ceased. The resulting mixture was diluted with EtOAc and the two layers were separated. The aqueous layer was extracted with EtOAc. The combined organic layers were washed with brine and dried with Na₂SO₄, filtered and concentrated. The crude residue was purified by prep-HPLC (acetonitrile/water/0.1% TFA). Fractions containing the product were free-based with aqueous NaHCO₃ and extracted with EtOAc. The organic layer was washed with brine and dried over Na₂SO₄, filtered and concentrated to give 10 mg (27%) of the title compound as a solid: ¹H NMR (DMSO-d₆) δ 9.36 (s, 1H), 9.17 (s, 1H), 8.6 (d, 1H), 7.85 (m, 2H), 7.38 (d, 1H), 7.14 (m, 3H), 7.02 (m, 2H), 6.78 (m, 1H), 6.7 (m, 1H), 6.24 (s, 2H), 5.9 (s, 1H), 2.84 (m, 2H), 2.63 (m, 2H); MS ES 468(M+H)⁺ calcd 468, RT=2.64 min.

By using the method described, and by substituting appropriate starting materials, examples 19 and 36 were also prepared.

### Example 39A

### 6-[(E)-2-(4-aminophenyl)vinyl]-N⁴-(4-{[2-(trifluoromethyl)pyridin-4-yl]oxy}phenyl)pyrimidine-2,4-diamine trifluoroacetate

### Example 39B

### 6-[(Z)-2-(4-aminophenyl)vinyl]-N⁴-(4-{[2-(trifluoromethyl)pyridin-4-yl]oxy}phenyl)pyrimidine-2,4-diamine trifluoroacetate

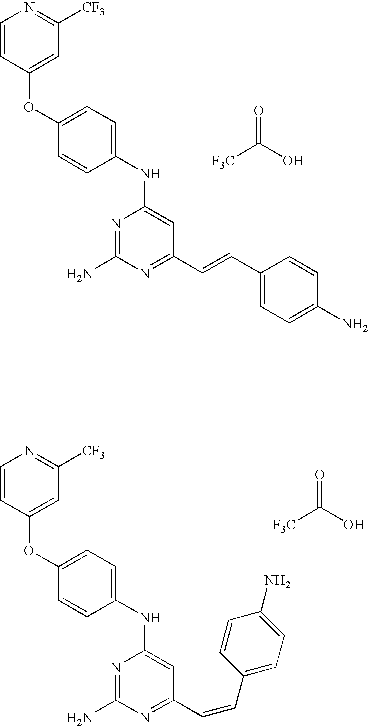

### Step 1: Preparation of 4,4,5,5-tetramethyl-2-[(E)-2-(4-nitrophenyl)vinyl]-1,3,2-dioxaborolane

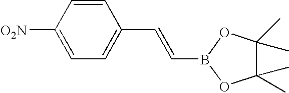

To a solution of 1-bromo-4-nitrobenzene (3.23 g, 16 mmol), palladium (II) acetate (0.22 g, 0.97 mmol) and Ph₃P (0.51 g, 1.95 mmol) in anhydrous toluene (70 mL) was added tri-n-butylamine (6.03 g, 32.53 mmol) and vinylboronic acid pinacol cyclic ester (3 g, 19.48 mmol). This mixture was then heated at 110° C. for 60 h. It was cooled to rt, diluted with EtOAc (120 mL), and stirred with 1N HCl (100 mL) for 10 min. The organic layer was separated, washed with water and brine, dried over Na₂SO₄, filtered, and concentrated to give a reddish-brown gum. The crude product was purified by silica gel chromatography (CH₂Cl₂) to afford 1.13 g (22%) of the desired product.

### Step 2: Preparation of 6-[(E)-2-(4-nitrophenyl)vinyl]-N⁴-(4-{[2-(trifluoromethyl)pyridin-4-yl]oxy}phenyl)pyrimidine-2,4-diamine

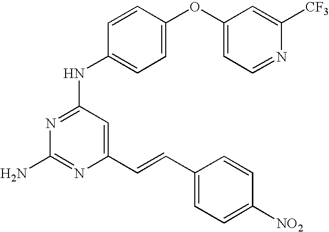

A mixture of 6-chloro-N⁴-(4-{[2-(trifluoromethyl)pyridin-4-yl]oxy}phenyl)pyrimidine-2,4-diamine (198 mg, 0.52 mmol), 4,4,5,5-tetramethyl-2-[(E)-2-(4-nitrophenyl)vinyl]-1,3,2-dioxaborolane (200 mg, 0.73 mmol), Na₂CO₃ (2.0 M, 0.91 mL), and PdCl₂dppf (85 mg, 0.10 mmol) in DMA (5 mL) was heated at 130° C. overnight before it was cooled to rt and concentrated. The residue was then purified by silica gel chromatography (4% MeOH in CH₂Cl₂) to afford 79 mg (31%) of the desired product. MS ES: 495.3 (M+H)⁺, calcd 494.1, RT=3.34 min.

### Step 3: Preparation of the Title Compounds

A mixture of 6-[(E)-2-(4-nitrophenyl)vinyl]-N⁴-(4-{[2-(trifluoromethyl)pyridin-4-yl]oxy}phenyl)pyrimidine-2,4-diamine (79 mg, 0.16 mmol) and SnCl₂.2H₂O (180 mg, 0.80 mmol) in EtOH (15 mL) was heated at 70° C. for 30 min before it was cooled to rt, filtered, and concentrated. The crude residue was purified by prep-HPLC to give 25.2 mg (34%) of the title compounds as a mixture of cis- and trans-olefin isomers. MS ES: 465.3 (M+H)⁺, calcd 464.1, RT=2.43 min.

### Example 40

### 6-[2-(4-aminophenyl)ethyl]-N⁴-(4-{[2-(trifluoromethyl)pyridin-4-yl]oxy}phenyl)pyrimidine-2,4-diamine trifluoroacetate

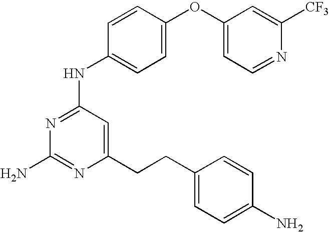

A mixture of 6-[(E)-2-(4-nitrophenyl)vinyl]-N-(4-{[2-(trifluoromethyl)pyridin-4-yl]oxy}phenyl)pyrimidine-2,4-diamine, 6-[(Z)-2-(4-nitrophenyl)vinyl]-N⁴-(4-{[2-(trifluoromethyl)pyridin-4-yl]oxy}phenyl)pyrimidine-2,4-diamine (80 mg, 0.16 mmol) and 10 wt % Pd on activated carbon (8 mg) in MeOH (16 mL) was stirred under an H₂ balloon for 4.5 h. It was then filtered and concentrated. The crude residue was purified by prep-HPLC to give 28 mg (30%) of the title compound as an off-white solid. MS ES: 467.3 (M+H)⁺, calcd 466.2, RT=2.16 min.

### Example 41

### N⁴-{4-[(2-methylpyridin-4-yl)oxy]phenyl}-6-[(E)-2-phenylvinyl]-pyrimidine-2,4-diamine

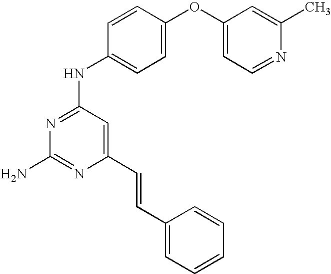

A mixture of 1 equivalent of 6-chloro-N⁴-{4-[(2-methylpyridin-4-yl)oxy]phenyl}pyrimidine-2,4-diamine, 2 equivalents of [(E)-2-phenylvinyl]boronic acid and 0.1 equivalent of PdCl₂(dppf)-CH₂Cl₂ complex in 2.5 mL anhydrous N,N-dimethylacetamide and 0.5 mL of 2 M K₂CO₃ in water in a 5 mL microwave reaction vessel under nitrogen was heated at 140° C. for 20 min in the personal microwave reactor (Emrys optimizer by Personal Chemistry). The reaction mixture was filtered, and the filtrate was concentrated and purified by prep-HPLC eluting with 15% to 85% acetonitrile containing 0.1% TFA using a Phenomenex Luna 5μ C18 150×30 mm column to provide the final product.

By using the method described, and by substituting appropriate starting materials, examples 42-46 were also prepared.

The examples are listed in the following table:

### B. PHYSIOLOGICAL ACTIVITY

The utility of the compounds of the present invention can be illustrated, for example, by their activity in vitro in the in vitro tumor cell proliferation assay described below. The link between activity in tumor cell proliferation assays in vitro and anti-tumor activity in the clinical setting has been very well established in the art. For example, the therapeutic utility of taxol (Silvestrini et al. Stem Cells 1993, 11(6), 528-35), taxotere (Bissery et al. Anti Cancer Drugs 1995, 6(3), 339), and topoisomerase inhibitors (Edelman et al. Cancer Chemotizer. Phanmacol. 1996, 37(5), 385-93) were demonstrated with the use of in vitro tumor proliferation assays.

The in vitro effect of the compounds according to the invention can be demonstrated in the following assays:

### Cytotoxic Activity of the Invention Compounds

The following section describes an assay that can be used to characterize compounds of the invention, e.g., to test for the cytotoxic activity of compounds on cells.

Human tumor cells, e.g., HCT116 cells, are seeded in a 96-well plate at 3.0×10³ cells/well and grown in 100 μl of RPMI complete media (Invitrogen Corporation, Grand Island, N.Y.) containing 10% fetal bovine serum (Hyclone, Logan, Utah) and 10 mM HEPES and at 37° C. for 16 h in an incubator with 5% CO₂. To each well, 50 μl of additional growth media containing 20 μM to 60 nM concentrations of compound with 0.2% DMSO is added. Cells are grown for another 72 h at 37° C. 20 μl of Alamar Blue (Trek Diagnostic Systems, Inc., Cleveland, Ohio) reagent is added to each well and incubated for 4 h at 37° C. Plates are read in a SpectraMax Gemini (Molecular Devices, CA) with 544 nm excitation and 590 nm emission wavelength. IC₅₀ values are determined by linear regression analysis of log drug concentration versus percent inhibition.

Representative compounds of this invention were tested for cytotoxicity using the above-described assay procedure with the following results:

Examples 3, 4, 5, 6, 7, 8, 15, 16, 18, 19, 20, 21, 22, 23, 26, 27, 28, 29, 30, 31, 32, 33, 34, 36, 37, 38, 40, 41, 44, 45, and 46 show an IC₅₀ of less than or equal to 500 nM in the HCT116 cytotoxic activity assay.

Examples 1, 2, 9, 10, 11, 12, 13, 14, 17, 24, 25, 35, 42, 43, 39A and 39B show an IC₅₀ greater than 500 nM but less than or equal to 10 μM in the HCT116 cytotoxic activity assay.

### C. OPERATIVE EXAMPLES RELATING TO PHARMACEUTICAL COMPOSITIONS

The compounds according to the invention can be converted into pharmaceutical preparations as follows:

### Tablet:

### Composition:

100 mg of the compound of Example 1, 50 mg of lactose (monohydrate), 50 mg of maize starch (native), 10 mg of polyvinylpyrrolidone (PVP 25) (from BASF, Ludwigshafen, Germany) and 2 mg of magnesium stearate.

Tablet weight 212 mg, diameter 8 mm, curvature radius 12 mm.

### Preparation:

The mixture of active component, lactose and starch is granulated with a 5% solution (m/m) of the PVP in water. After drying, the granules are mixed with magnesium stearate for 5 min. This mixture is moulded using a customary tablet press (tablet format, see above). The moulding force applied is typically 15 kN.

### Suspension for Oral Administration:

### Composition:

1000 mg of the compound of Example 1, 1000 mg of ethanol (96%), 400 mg of Rhodigel (xanthan gum from FMC, Pennsylvania, USA) and 99 g of water.

A single dose of 100 mg of the compound according to the invention is provided by 10 ml of oral suspension.

### Preparation:

The Rhodigel is suspended in ethanol and the active component is added to the suspension. The water is added with stirring. Stirring is continued for about 6 h until the swelling of the Rhodigel is complete.

### Solution for Intravenous Administration 1:

Composition: 100-200 mg of the compound of Example 1, 15 g polyethylenglykol 400 and 250-g water. in saline optionally with up to 15% Cremophor EL, and optionally up to 15% ethyl alcohol, and optionally up to 2 equivalents of a pharmaceutically suitable acid such as citric acid or hydrochloric acid.

### Preparation:

The compound of Example 1 and the polyethylenglykol 400 are dissolved in the water with stirring. The solution is sterile filtered (pore size 0.22 μm) and filled into heat sterilized infusion bottles under aseptical conditions. The infusion bottles are being sealed with rubber seals.

### Solution for Intravenous Administration 2:

Composition: 100-200 mg of the compound of Example 1, saline solution, optionally with up to 15% by weight of Cremophor EL, and optionally up to 15% by weight of ethyl alcohol, and optionally up to 2 equivalents of a pharmaceutically suitable acid such as citric acid or hydrochloric acid.

### Preparation:

The compound of Example 1 is dissolved in the saline solution with stirring. Optionally Cremophor EL, ethyl alcohol or acid are added. The solution is sterile filtered (pore size 0.22 μm) and filled into heat sterilized infusion bottles under aseptical conditions. The infusion bottles are being sealed with rubber seals.

Other embodiments of the invention will be apparent to the skilled in the art from a consideration of this specification or practice of the invention disclosed herein. It is intended that the specification and examples be considered as exemplary only, with the true scope and spirit of the invention being indicated by the following claims.

1. A compound of formula (I) 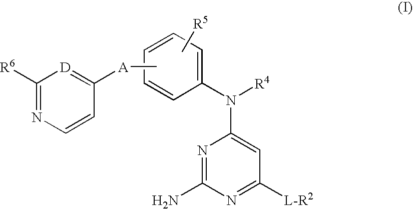
wherein
A represents an oxygen atom or a group —NR^{A}—, in which R^{A} represents hydrogen or alkyl;
D represents a group —CH— or a nitrogen atom;
L is a 2 carbon atom linker selected from the group consisting of ethandiyl, ethendiyl and ethyndiyl, which in case of ethandiyl can optionally be substituted by 0, 1 or 2 alkyl, hydroxy or alkoxy, in case of ethendiyl can optionally be substituted by 0, 1 or 2 alkyl or alkoxy;
R² represents alkyl, wherein alkyl can be substituted with 0 to 3 substituents selected from the group consisting of halo, hydroxy, alkoxy, amino, alkylamino, and alkylsulfonylamino; or
R² represents phenyl or heteroaryl, wherein phenyl or heteroaryl can optionally be substituted by 0, 1 or 2 substituents selected from the group consisting of halo, trifluoromethyl, alkyl, hydroxy, alkoxy, amino, alkylcarbonylamino, alkylamino, aminocarbonyl, alkylaminocarbonyl, aminosulfonyl, alkylarinosulfonyl, and,
wherein said alkylamino, alkylaminocarbonyl, and alkylaminosulfonyl are optionally substituted by 0, 1 or 2 substituents selected from the group consisting of hydroxy, halogen and alkoxy;
R⁴ is hydrogen or alkyl;
R⁵ is hydrogen or halo; and
R⁶ represents alkyl, cyano, aminocarbonyl, alkylaminocarbonyl, trifluoromethyl, amino, alkylcarbonylamino, alkylcarbonyl, alkenyl, alkynyl or chloro;
or a pharmaceutically acceptable salt thereof.

2. The compound of claim 1, wherein
A represents an oxygen atom;
D represents a group —CH—;
L is a 2 carbon atom linker selected from the group consisting of ethandiyl, ethendiyl and ethyndiyl;
R² represents alkyl, wherein alkyl can be substituted with 0 to 2 substituents selected from the group consisting of halo, hydroxy, alkoxy, amino, alkylamino, and alkylsulfonylamino; or
R² represents phenyl or pyridyl, wherein phenyl or pyridyl can optionally be substituted by 0, 1 or 2 substituents selected from the group consisting of halo, alkyl, hydroxy, and alkoxy;
R⁴ is hydrogen;
R⁵ is hydrogen; and
R⁶ represents alkyl, cyano, aminocarbonyl, chloro or trifluoromethyl;
or a pharmaceutically acceptable salt thereof.

3. A compound of formula (Ia), 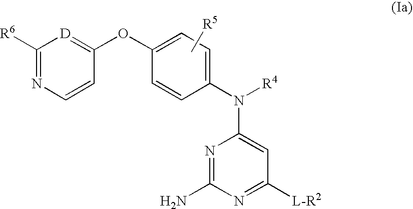
wherein
D represents a group —CH—;
L is a 2 carbon atom linker selected from the group consisting of ethandiyl, ethendiyl and ethyndyl;
R² represents phenyl or pyridyl, wherein phenyl or pyridyl can optionally be substituted by 0, 1 or 2 substituents selected from the group consisting of halo, alkyl, hydroxy, and alkoxy;
R⁴ is hydrogen;
R⁵ is hydrogen; and
R⁶ represents alkyl, cyano, aminocarbonyl, chloro or trifluoromethyl;
or a pharmaceutically acceptable salt thereof.

4. A process for preparing a compound of claim 1, comprising reacting
a compound of formula (II) 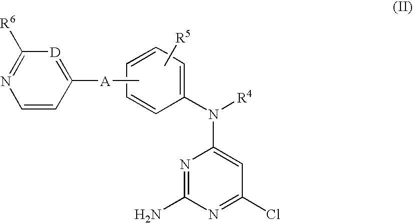
[A] with an agent of formula (IIIa) 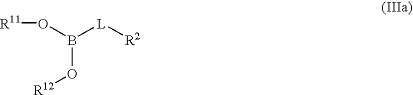
in which L and R² have the meaning indicated above, and R¹¹ and R¹² can be H or alkyl, or
[B] with an agent of formula (IIIb) 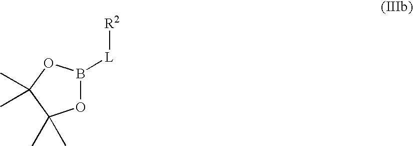
in which L and R² have the meaning indicated above, or
[C] with an agent of formula (IIIc) 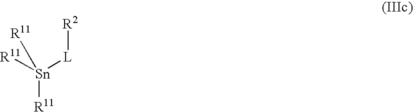
in which L, R² and R¹¹ have the meaning indicated above, in the presence of a suitable Pd catalyst.

5. The compound of claim 1 for the treatment or prevention of disorders.

6. A pharmaceutical composition comprising the compound of claim 1.

7. The pharmaceutical composition of claim 6, additionally comprising at least one pharmaceutically acceptable carrier or excipient.

8. The pharmaceutical composition of claim 6, in a form suitable for oral or intravenous administration.

9. The pharmaceutical composition of claim 6 for the treatment or prevention of disorders.

10. A process for preparing the pharmaceutical composition of claim 7, comprising combining at least one compound according to claim 1 with at least one pharmaceutically acceptable carrier or excipient and bringing the resulting combination into a form suitable for said pharmaceutical composition.

11. The use of a compound of claim 1 for manufacturing a pharmaceutical composition for the treatment or prevention of disorders.

12. The use of claim 11, wherein the disease is a cancer.

13. A method of treating a disease or condition in a mammal, comprising administering to a mammal in need thereof an effective amount of a compound of claim 1.

14. The method of claim 13, wherein the disease or condition is cancer.

15. A packaged pharmaceutical composition comprising a container comprising the pharmaceutical composition of claim 7 and instructions for using the pharmaceutical composition to treat a disease or condition in a mammal.
